SEQUENCE LISTING (15 sequences)

SEQ ID NO: 1 - DNA, 42 bp, synthetic construct
  source 1..42: mol_type = other DNA
gacgacgagtagcgcgtatgaatgcttttctatggagtcgtc

SEQ ID NO: 2 - DNA, 36 bp, synthetic construct
  source 1..36: mol_type = other DNA
gacgactggagttttaacggctcttgcgaagtcgtc

SEQ ID NO: 3 - DNA, 47 bp, synthetic construct
  source 1..47: mol_type = other DNA
ctctcgacgacgagtagcgcgtatgaatgcttttctatggagtcgtc

SEQ ID NO: 4 - DNA, 41 bp, synthetic construct
  source 1..41: mol_type = other DNA
ctctcgacgactggagttttaacggctcttgcgaagtcgtc

SEQ ID NO: 5 - DNA, 47 bp, synthetic construct
  source 1..47: mol_type = other DNA
ctctcgacgaccgtgtgtgttgctctgtaacagtgtccattgtcgtc

SEQ ID NO: 6 - DNA, 12 bp, synthetic construct
  source 1..12: mol_type = other DNA
agtcgtcgagag

SEQ ID NO: 7 - DNA, 92 bp, synthetic construct
  source 1..92: mol_type = other DNA
aatgatacggcgaccaccgagatctacactagatcgcacactctttccctacacgacgctcttccgatctttacgattgcagcatcgggacg

SEQ ID NO: 8 - DNA, 84 bp, synthetic construct
  source 1..84: mol_type = other DNA
caagcagaagacggcatacgagatctagtacggtgactggagttcagacgtgtgctcttccgatctggaggctctcgggacgac

SEQ ID NO: 9 - DNA, 84 bp, synthetic construct
  source 1..84: mol_type = other DNA
caagcagaagacggcatacgagatttctgcctgtgactggagttcagacgtgtgctcttccgatctggaggctctcgggacgac

SEQ ID NO: 10 - DNA, 84 bp, synthetic construct
  source 1..84: mol_type = other DNA
caagcagaagacggcatacgagatgctcaggagtgactggagttcagacgtgtgctcttccgatctggaggctctcgggacgac

SEQ ID NO: 11 - DNA, 72 bp, synthetic construct
  source 1..72: mol_type = other DNA
ggaggctctcgggacgacnnnnnnnnnnnnnnnnnnnnnnnnnnnnnngtcgtcccgatgctgcaatcgtaa

SEQ ID NO: 12 - DNA, 18 bp, synthetic construct
  source 1..18: mol_type = other DNA
ggaggctctcgggacgac

SEQ ID NO: 13 - DNA, 22 bp, synthetic construct
  source 1..22: mol_type = other DNA
ttacgattgcagcatcgggacg

SEQ ID NO: 14 - DNA, 22 bp, synthetic construct
  source 1..22: mol_type = other DNA
ttacgattgcagcatcgggacg

SEQ ID NO: 15 - DNA, 18 bp, synthetic construct
  source 1..18: mol_type = other DNA
gtcgtcccgagagccata

(19) United States
(12) Patent Application Publication
(10) Pub. No.: US 2025/0298026 A1
(43) Pub. Date: Sep. 25, 2025

(54) DNA Aptamers for Detection of L-Lactate and Methods Therefor

(71) Applicants: Juewen LIU, Waterloo (CA); Po-Jung Jimmy HUANG, Waterloo (CA)

(72) Inventors: Juewen LIU, Waterloo (CA); Po-Jung Jimmy HUANG, Waterloo (CA)

(21) Appl. No.: 19/103,485

(22) PCT Filed: Aug. 11, 2023
(86) PCT No.: PCT/CA2023/051072

§ 371 Date: Feb. 12, 2025

Related U.S. Application Data
(60) Provisional application No. 63/474,381, filed on Aug. 12, 2022.

(51) Int. Cl. G01N 33/66; C12N 15/115; G01N 21/64; G01N 33/53

(52) U.S. Cl. CPC G01N 33/66; C12N 15/115; G01N 21/6428; G01N 33/5308; C12N 2310/16

(57) ABSTRACT

Aptamers specific for L-lactate are provided. Also provided are biosensors comprising such aptamers and methods for detecting L-lactate using such aptamers and/or biosensors. Also provided is a method for multiplexed detection of L-lactate and D-glucose.

### CROSS REFERENCE TO PRIOR APPLICATIONS

The present application claims priority to U.S. Application No. 63/474,381, filed Aug. 12, 2023, the entire contents of which are incorporated herein by reference.

### STATEMENT REGARDING SEQUENCE LISTING

A Sequence Listing associated with this application is electronically submitted herewith in XML format. The file containing the Sequence Listing is entitled “SeqList-53813-00106.XML”, was created on Aug. 11, 2023. The entire contents of the Sequence Listing are incorporated by reference into the present specification.

### FIELD OF THE DESCRIPTION

The present description relates to DNA aptamers for L-lactate. In one aspect, the description relates to biosensors comprising such aptamers and to methods of detecting L-lactate in a sample using the aptamers and/or biosensors.

### BACKGROUND

Under conditions of limited oxygen, anaerobic glycolysis results in the conversion of D-glucose (or “glucose” as used herein) to L-lactate (or “lactate” as used herein). L-glucose is not naturally occurring, and D-lactate is produced by some bacterial strains [1]. Lactate concentration in blood normally ranges from 0.5 to 2.2 mM, although this concentration increases in a variety of physiological and pathological conditions. For example, lactate serum concentration can spike to over 20 mM during intense physical exercise [2]. For this reason, athletes often monitor serum lactate concentration in order to stay in an aerobic metabolic state. While lactate is quickly metabolized to pyruvate, its accumulation is often indicative of pathological states. For example, a high concentration of lactate may be an indication of sepsis and/or other critical conditions, so a detection of high lactate concentration can predict mortality [3]. Measurement of lactate concentration is also important in the food industry, for monitoring fermentation processes and/or to detect bacterial contamination. For example, lactate concentration measurements are often used to monitor wine production [4, 5] and to estimate water contamination [6-7].

The detection of lactate has been traditionally carried out using enzymes, namely lactate oxidase (LOx) and lactate dehydrogenase (LDH) [4, 8-10]. These known methods have associated deficiencies. LOx, for example, produces H₂O₂ as a co-product, which also occurs with glucose oxidase (GOx) and many other enzymes. This leads to inaccuracies in 4 lactate detection, especially during multiplexed detection methods with a high spatial resolution. LDH requires NAD+ as an electron acceptor. The reliability and sensitivity of such enzyme-based biosensors is affected by enzyme activity, denaturation, and the concentration of oxygen or NAD+, which makes calibration difficult.

Owing to the correlations between lactate (L-lactate) and glucose (D-glucose) to metabolic conditions, it is highly desirable to concurrently monitor glucose and lactate concentrations. Such monitoring is particularly effective during exercise [2], or for detecting diabetic lactic acidosis or cancer [12]. Various biosensors have been reported for such concurrent detection of lactate and glucose, but these are all based on the aforementioned enzymes [13-18].

Aptamers are single-stranded DNA molecules that have been shown to bind to a diverse range of target molecules with high affinity and specificity [19-24]. Unlike enzymes, aptamers rely solely on binding for target recognition and are independent of other reactants such as oxygen or NAD+ [25], making them ideal for continuous monitoring. U.S. Pat. No. 7,745,607 teaches a method of selecting aptamers. U.S. Pat. Nos. 8,541,561 and 9,000,137 describe aptamers that are specific to certain analytes.

Lactate and glucose are both low epitope molecules and it has long been perceived to be difficult to obtain aptamers for these molecules [26]. Fortunately, these two molecules are present in mM concentrations in blood, and thus aptamers in this affinity range are sufficient. An aptamer for glucose was reported by the Stojanovic group with a Kd of 10 mM [27]. Isolation of aptamers for lactate appeared to be even more challenging since it has only three carbons and is a negatively charged molecule. Thus, no aptamers for lactate are known to date.

### SUMMARY OF THE DESCRIPTION

In one aspect, there is provided a DNA aptamer for binding to L-lactate, the aptamer comprising a nucleic acid sequence according to SEQ ID NO: 1 or SEQ ID NO: 2, or a sequence having at least 80% homology thereto.

In another aspect, there is provided a biosensor for detecting the presence of L-lactate, the biosensor comprising the aptamer of SEQ ID NO: 1 or SEQ ID NO: 2, or a sequence having at least 80% homology thereto.

In another aspect, there is provided a method of detecting or monitoring the presence of L-lactate in a sample, the method comprising the use of an aptamer or biosensor described herein that is specific to L-lactate. In one preferred aspect, the aptamer is the aptamer having a nucleic acid sequence of SEQ ID NO: 1 or SEQ ID NO: 2 or the biosensor having the nucleic acid sequence of SEQ ID NO: 3 or SEQ ID NO: 4. In one aspect, the binding of the aptamer or biosensor to L-lactate emits a signal indicative of he presence of L-lactate in the sample. In one aspect, the sample is blood.

In another aspect, there is provided a method of multiplexed or simultaneous detection of L-lactate and D-glucose in a sample, wherein the method comprises the use of an aptamer having a nucleic acid sequence of SEQ ID NO: 1 or SEQ ID NO: 2 or a biosensor having the nucleic acid sequence of SEQ ID NO: 3 or SEQ ID NO: 4, along with an aptamer or biosensor specific to D-glucose.

Other methods and kits are provided.

### BRIEF DESCRIPTION OF THE FIGURES

The features of certain embodiments will become more apparent in the following detailed description in which reference is made to the appended figures wherein:

FIG. 1 illustrates possible secondary structures of Lac201 predicted by Mfold™. The structure shown on the right is the correct structure based on sequence alignment.

FIG. 2A Illustrates the sequence alignment of the first 10 most abundant sequences in the round 20 library and their copy number out of a total of obtained 51,152 sequences.

FIG. 2B to 2D illustrate the predicted secondary structures of the Lac201 (FIG. 2B), Lac204 (FIG. 2C), and Lac202 (FIG. 2D) aptamers. 2

FIG. 3 illustrates the alignment of all the Family 3 sequences from the top 70 sequences. The number of copy of each sequence in the final sequenced library is also shown.

FIG. 4A illustrates the scheme of the fluorescent biosensor for lactate detection.

FIGS. 4B-4E illustrate the kinetics of sensor signaling using Lac201 (FIG. 4B) and Lac202 (FIG. 4D). Lactate was added at 5 min. Change of fluorescence as a function of L-lactate and D-lactate concentration using the Lac201 (FIG. 4C) and Lac202 (FIG. 4E) aptamers.

FIGS. 5A and 5B illustrate the fluorescence quenching of 5 nM of FAM-Lac201 (FIG. 5A) and FAM-Lac202 (FIG. 5B) sensors with increasing concentration of Quencher-cDNA in SELEX™ buffer. This experiment allows us to obtain the Kd of the original aptamer based on Easley's work [46].

FIGS. 6A and 6B illustrate the kinetic trace of FAM-Lac201 (FIG. 6A) and FAM-Lac202 (FIG. 6B) sensors with various concentration of D-lactate in the SELEX™ buffer.

FIG. 7 illustrates the selectivity of the Lac201 sensor against certain biomolecules in serum. The structure and normal physiological concentration range of these analytes are also shown (from known values [43]).

FIG. 8 illustrates the selectivity of the Lac202 sensor against certain biomolecules in SELEX™ buffer.

FIGS. 9A-9D illustrate the kinetics traces of 20 nM Lac201 (FIG. 9A) and Lac202 (FIG. 9C) sensors in the presence of various concentrations of lactate in 10% human serum. Lactate was added at the 5 min mark. FIGS. 9B and 9D show the fluorescence response of each sensor as a function of lactate concentration.

FIGS. 10A and 10B illustrate glucose sensor performance in SELEX™ buffer. FIG. 10A illustrates the kinetic trace of 20 nM sensor in different concentrations of glucose. FIG. 10B illustrates the fluorescence intensity as a function of glucose concentration. The inset in FIG. 10B illustrates a linear response at low glucose concentrations. 2

FIG. 11A illustrates a scheme showing simultaneous detection of glucose and lactate by mixing the two aptamer-based biosensors with different fluorophores.

FIG. 11B illustrates time-dependent responses of the two sensors in the same 10% human serum solution.

FIG. 11C is a photograph of the sensor when adding lactate and glucose.

FIG. 12 shows the results of an isothermal titration calorimetry (ITC) thermogram and integrated heat of titrating 20 mM L-lactate to 300 UM Lac-201 at 20° C. in SELEX™ 9 buffer.

### DETAILED DESCRIPTION

As used herein, the term “lactate” will be understood to mean L-lactate unless otherwise stated. Similarly, the term “glucose” will be understood to mean D-glucose unless otherwise stated.

The terms “sensor” or “biosensor” may be used herein interchangeably. These terms are intended to refer to an aptamer that is adapted to provide a signal once bound to a target analyte. In one aspect, a sensor (or biosensor) may comprise an aptamer to which is attached a fluorescent moiety, adapted to fluoresce once the aptamer is bound to the target analyte.

The term “signal” as used herein may comprise a visual or electrical signal that is emitted upon a detector molecule binding to the analyte to which it is specific. Although the present description teaches a visual signal, comprising a fluorescence, it will be understood that the description is not limited to such signal.

Each of the terms “sequence identity”, “similarity”, or “homology”, as may be used herein, refers to the degree to which two polynucleotide sequences are identical on a residue-by-residue basis over a particular region of comparison. The degree, or percentage of sequence identity, similarity, or homology is calculated by comparing two optimally aligned sequences over a region of comparison, determining the number of positions at which the identical nucleic acid base (e.g., A, T, C, G) occurs in both sequences to yield the number of matched positions, dividing the number of matched positions by the total number of positions in the region of comparison (i.e., the window size), and multiplying the result by 100 to yield the percentage of sequence identity. The term “substantial identical” or “homolog” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. Therefore, the terms “substantially identical” or “homolog” as used herein will be understood as being a characteristic of a polynucleotide sequence that has at least 80%, 81%, 82%, 83%, 84%, 85%, 86%, 87%, 88%, 89%, 90%, 91%, 92%, 93%, 94%, 95%, 96%, 97%, 98%, or 99% sequence identify as compared to a reference sequence over the specified comparison region.

The terms “comprise”, “comprises”, “comprised” or “comprising” may be used in the present description. As used herein (including the specification and/or the claims), and unless stated otherwise, these terms are to be interpreted as open-ended terms and as specifying the presence of the stated features, integers, steps or components, but not as precluding the presence of one or more other feature, integer, step, component or a group thereof as would be apparent to persons having ordinary skill in the relevant art. Thus, the term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

The phrase “consisting essentially of” or “consists essentially of” will be understood as generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open-ended term, such as “comprising” or “including”, it will be understood that direct support should be afforded also to “consisting essentially of” language as well as “consisting of” language as if stated explicitly and vice versa. In essence, use of one of these terms in the specification provides support for all of the others.

For the purposes of the present specification and/or claims, and unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth herein are approximations that may vary depending upon the desired properties sought to be obtained by the present invention, inclusive of the stated value and has the meaning including the degree of error associated with measurement of the particular quantity. The term “about” generally refers to a range of numbers that one of ordinary skill in the art would consider as a reasonable amount of deviation to the recited numeric values (i.e., having the equivalent function or result). For example, this term “about” can be construed as including a deviation of +10 percent of the given numeric value provided such a deviation does not alter the end function or result of the value. Therefore, a value of about 1% may be construed to be a range from 0.9% to 1.1%.

The term “and/or” can mean “and” or “or”.

Unless stated otherwise herein, the articles “a” and “the”, when used to identify an element, are not intended to constitute a limitation of just one and will, instead, be understood to mean “at least one” or “one or more”.

As described further below, we performed an aptamer selection experiment to isolate DNA aptamers for lactate. We then designed fluorescent biosensors for the detection of lactate. Finally, simultaneous detection of both lactate and glucose in a single serum solution was achieved using the two sensors labeled with different fluorophores.

L-lactate is a key metabolite indicative of metabolic state, glycolysis pathways, and various diseases such as sepsis, heart attack, and cancer. Detection of lactate has been relying on a few enzymes that need other substrates. In this work, DNA aptamers for L-lactate were obtained using a library-immobilization selection method and two types of aptamers were obtained, identified herein as Lac201 and Lac202, with Kd values of 0.39 mM and 1.92 mM, respectively. These aptamers showed up to 50-fold selectivity for L-lactate over D-lactate and had no measurable response to other closely related analogs such as pyruvate and 3-hydroxybutyrate. Two fluorescent biosensors based on the strand displacement method showed limit of detection of 0.55 mM and 1.17 mM, respectively. Simultaneous detection of L-lactate and D-glucose using two fluorophore-labeled sensors in the same serum solution was achieved. This work has broadened the scope of aptamers to very simple metabolites and provided a useful probe for the continuous and multiplexed monitoring.

### Aptamers

As discussed further herein, the description provides unique aptamers having the nucleic acid sequences of SEQ ID NO: 1 or SEQ ID NO: 2, which specifically bind L-lactate.

### Biosensors

Also provided herein are biosensors for detecting the presence of L-lactate that comprise the aforementioned aptamers of SEQ ID NO: 1 or SEQ ID NO: 2. The biosensors further comprise a signalling moiety that is adapted to emit a signal once the aptamer binds to L-lactate. In one aspect, the signalling moiety emits a visual signal, such as a fluorescence. In one aspect, the signalling moiety is fluorescein. Preferred lactate biosensors comprise the nucleic acid sequences of SEQ ID NO: 3 or SEQ ID NO: 4.

In another aspect, there is provided a biosensor for detecting the presence of D-glucose. In one aspect, the glucose biosensor comprises the nucleic acid sequence of SEQ ID NO: 5.

### Methods

The description also provides methods of detecting or monitoring the presence of L-lactate in a sample comprising the use of the aptamers or biosensors described herein.

In another aspect, there is provided a method multiplexed, or simultaneous, detection or monitoring of L-lactate and D-glucose comprising the use of the aptamers or biosensors described herein. It will be understood that the signal emitted by the aptamer or biosensor specific to L-lactate will be different to the signal emitted by the aptamer or biosensor specific to D-glucose.

### Kits

In another aspect, the description provides kits that can be used for detecting the presence of L-lactate or the simultaneous detection of L-lactate and D-glucose. The kits may comprise, for example, a container for collecting samples and one or more of the aptamers or biosensors described herein. The kits may also include, without limitation, means for collecting samples to be assayed, solid supports, buffers, preservatives, standards, diluents, and/or instructions for carrying out a method as described herein. The solid support may comprise, for example, a microtiter multi-well plate, a test strip or beads 8 (onto which the one or more aptamers or biosensors may be immobilized). The kits may also include one or protease inhibitors (e.g., a protease inhibitor cocktail) for use with a biological sample to be assayed (such as blood, urine, etc.) The present description is not limited to any kit.

### Results and Discussion

We used the library immobilization method by hybridizing our DNA library containing a 30-nt random region (N₃₀) to a biotinylated DNA attached to streptavidin beads. After incubation with L-lactate, the binding sequences were collected and amplified by PCR [27-33]. Our aptamer selection was performed for a total of 20 rounds, during which the lactate concentration was gradually reduced from 1 M to 0.5 mM. The round 14, 18, 19 and 20 round libraries were deep sequenced. We aligned the 10 most abundant sequences in round 20 and found that they can be divided in three families. Family 1 represented by Lac201 has two conserved regions that switched side compared to Family 2 represented by Lac204. This has been commonly observed in small molecule binding aptamers such as the aptamers for theophylline and the DNA aptamer for uric acid [34]. This indicates the two orientations of lactate for the same way of binding. Family 3 represented by Lac202 has a different set of conserved sequences, which suggests a different way of binding.

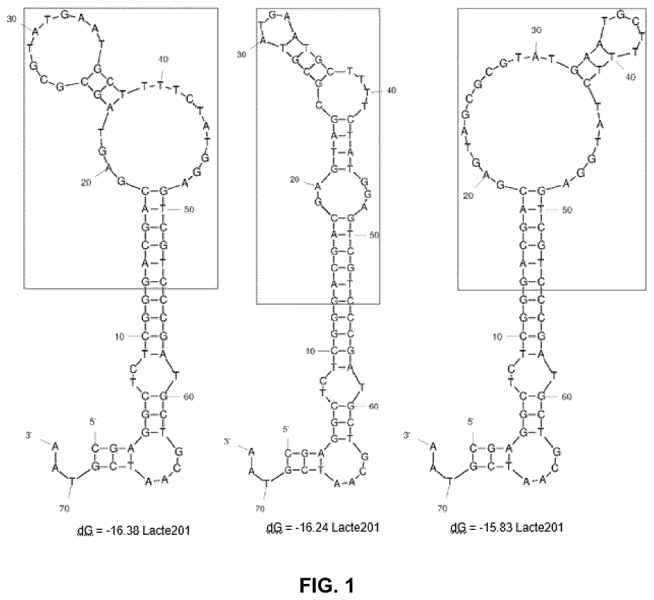

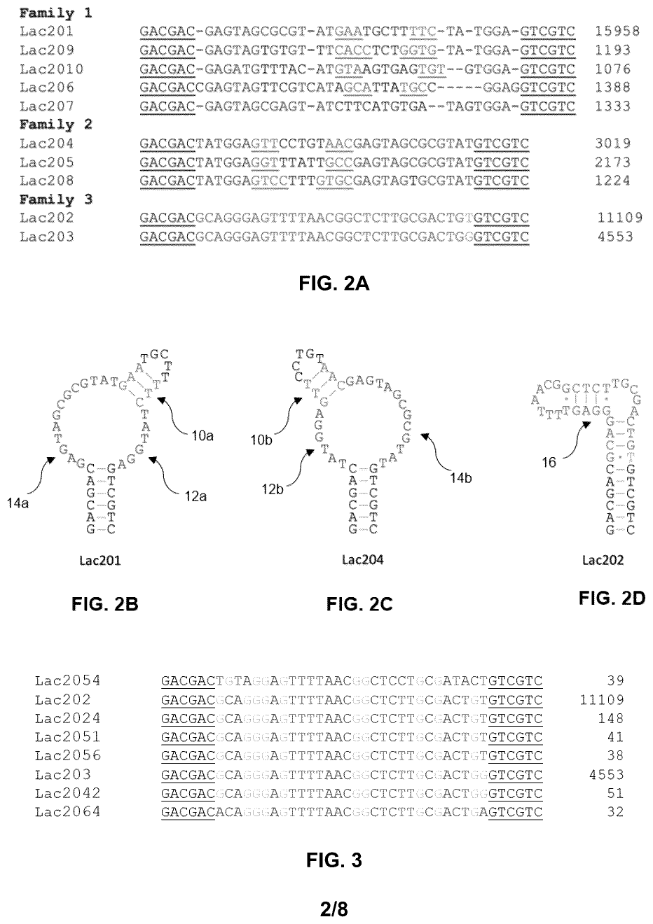

By comparing these sequences from different rounds, we found that Lac201 gradually decreased from round 14 to 20, while Lac202 gradually increased. Overall, even the round 14 library was quite converged indicating a successful selection. Three secondary structure of Lac201 were predicted by Mfold™ (FIG. 1) [37]. We reasoned that the structure shown in FIG. 2B was the correct folding since it can satisfy the sequence alignment within this family. The green region (shown at 10a) covaried to form a short 3-base-pair stem and the two conserved regions (shown in blue, at 12a, and red, at 14a) are on the two sides of this short stem. The folded structure of Lac204, FIG. 2C, has the conserved nucleotides, shown at 12b and 14b, but on switched sides and thus this structure was considered to have the same binding mechanism as Lac201 (FIG. 2B). Family 3 appeared 8 times in the top 70 sequences, and seven of them were found to have the exact 29-nt sequence, marked in purple and shown at 16 in FIG. 2A and shown in FIG. 3. Based on Mfold™, the predicted bulged two-way junction structure of Lac202 is shown in FIG. 2D.

To test aptamer binding to lactate, we performed isothermal titration calorimetry (ITC). As shown in FIG. 12, when lactate was titrated into Lac201 aptamer, an exothermic profile was observed with a fitted Kd of 0.43 mM, which is near the physiological level of lactate. Thus, this aptamer was determined to be useful for measuring lactate concentration fluctuation. In particular, considering the simple structure of lactate, the measured binding affinity was deemed to be acceptable. For comparison, and as discussed herein, the glucose aptamer has a Kd of 10 mM. In the course of the study, it was found that both enthalpy and entropy contributed to the binding, and the entropy contribution even dominated. A positive entropy change would be advantageous for the use of this aptamer at higher temperatures (e.g., body temperature).

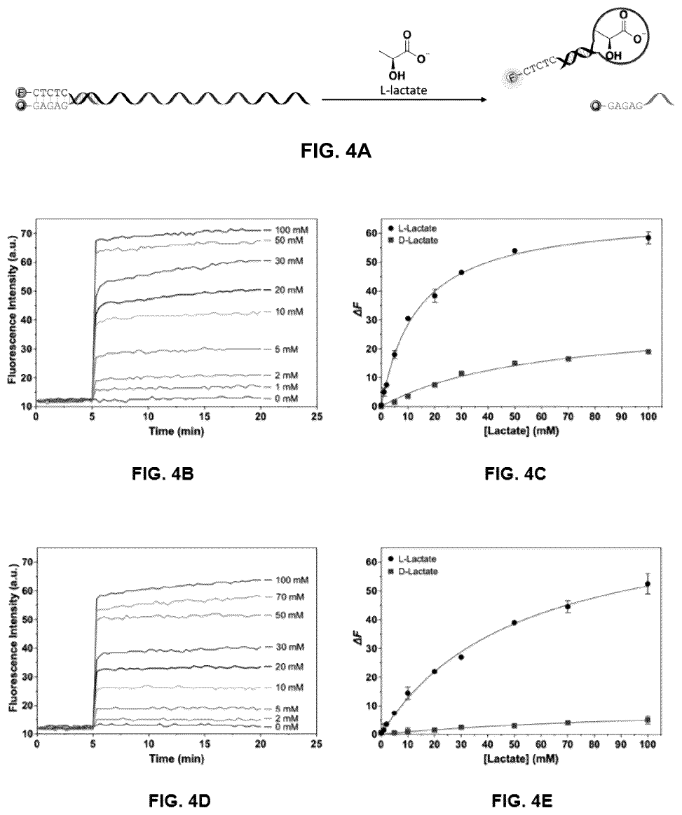

After confirming binding, we designed a biosensor for the detection of lactate. We extended the Lac201 and Lac202 aptamers by 5-nt on the 5′-end and labeled them with a FAM fluorophore. They were then hybridized with a 12-mer quencher-labeled DNA resulting in quenched fluorescence (FIG. 4A). Upon the addition of lactate, aptamer binding would release the quencher-labeled strand to produce enhanced fluorescence [36]. These two sensors were then incubated with different concentrations of lactate and indeed lactate-dependent fluorescence enhancement was observed (FIGS. 4B, 4D). Both sensors had an instantaneous fluorescence increase and the fluorescence immediately reached a stable value, and both sensors reached around 5-fold of fluorescence increase compared to the background. We then plotted the fluorescence enhancement as a function of lactate concentration (FIGS. 4C, 4E). Lac201 has a dynamic range up to 30 mM and a limit of detection (LOD) of 0.55 mM, which nicely covers the physiological range of lactate. For five commercial lactate biosensors based on LOx, none was found capable of covering the full range from 1 to 23 mM [37]. Therefore, our aptamer sensors are effective in the detection range.

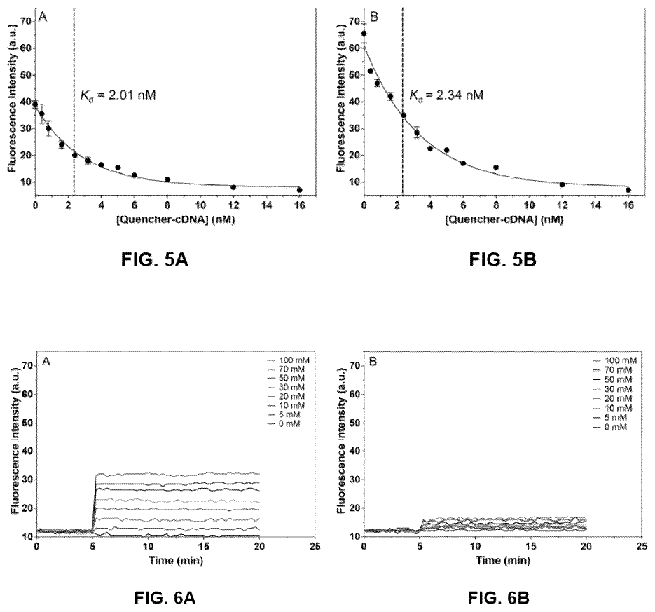

Although the apparent Kd of Lac201 from the sensor data was 13.5 mM, its actual Kd was calculated to be 0.39 mM after considering the competing effect of the quencher-labeled strand (FIGS. 5A and 5B) [36, 38]. This Kd is quite reasonable for such a small molecule as lactate. The glucose binding aptamer has a Kd of 10 mM [27]. By comparing the structure of glucose and lactate, the carboxylate in lactate likely promoted binding via metal-mediated interactions. We then tested the response to D-lactate, which also showed an instantaneous response (FIG. 6). At a concentration of 100 mM, the response was still far from saturation (see FIG. 4C) and an accurate Kd could not be fitted. The response to 20 mM D-lactate was similar to 2 mM L-lactate, and thus the selectivity was around 10-fold.

We then fitted the sensor response of Lac202, which has an even broader range up to 100 mM, and its LOD was calculated to be 1.2 mM. The real Kd was 1.92 mM. Its selectivity for L-lactate 5 over D-lactate was even better. 100 mM D-lactate response was similar to 2 mM of L-lactate (around 50-fold selectivity). The difference in the chiral selectivity of these two aptamers also indicated their different ways of binding to lactate. The chiral selectivity of aptamers is well documented [39, 40]. While lactate is a very simple molecule, it still showed good selectivity. The chiral selectivity is analytically useful since most instrumentation have difficulty in distinguishing L- and D-lactate [1]. Since D-lactate exists in blood only in patients with some rare diseases its interference is not believed to be a concern for the detection of L-lactate.

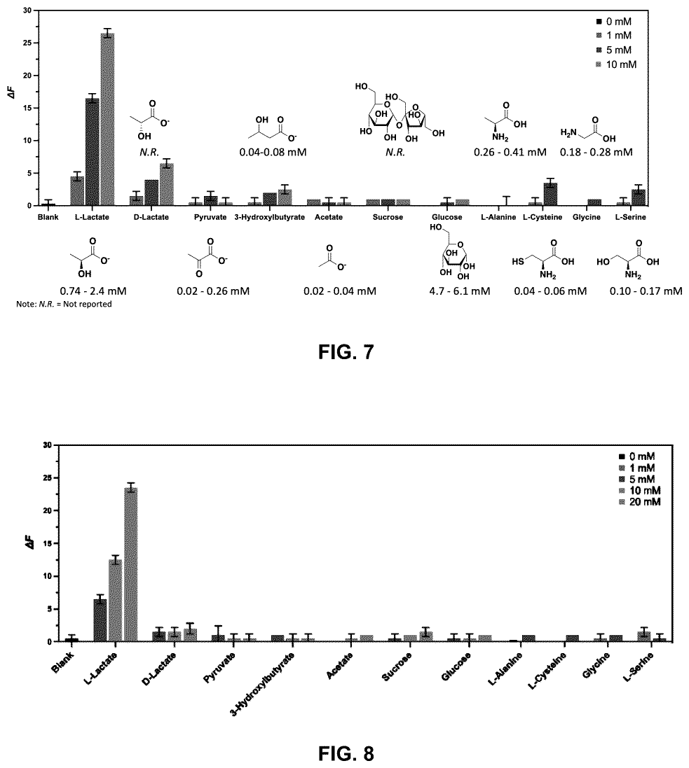

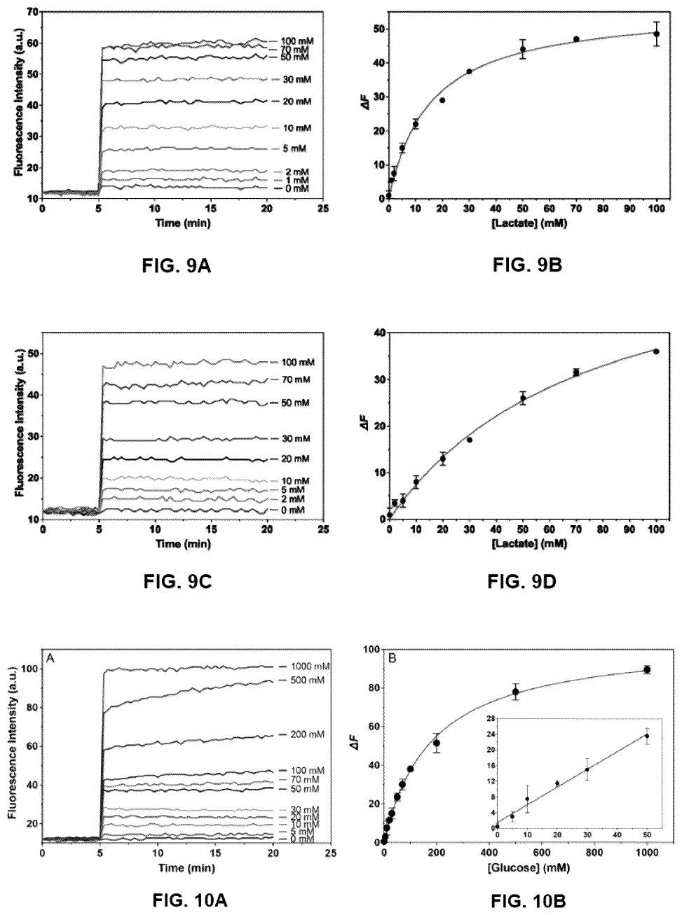

After confirming binding and sensitivity of the sensors, we then tested the selectivity against some related compounds, especially those might be present in blood (FIG. 7). We focused on Lac201 as it has a more sensitive response. Pyruvate is an important analyte since it is the product of lactate oxidation. 3-Hydroxybutyrate differs from lactate only by a single methylene group and it is a main keto body [42]. None of these showed any response. Acetate being a carboxylic acid, also showed no response. Among the two sugars and four amino acids, only cysteine and serine showed a slight response at the 5 mM level. Other than lactate and glucose, all the other tested molecules are only in μM concentration (FIG. 7 indicates the physiological range of these molecules below the respective structures) [43], yet they showed no response even at 1 mM. Even better selectivity was achieved for the Lac202 sensor (FIG. 8). Overall, for the analysis of lactate in blood or serum, the sensitivity and selectivity of Lac201 are sufficient.

Since the detection of lactate in blood serum is critical for its application, we then tested the Lac201 sensor in 10% human serum (FIGS. 9A-9D). The response was very similar to that in buffer and we still obtained a detection range of up to 30 mM and a limit of detection of 1.4 mM. Therefore, serum proteins had little effect on the performance of the sensor.

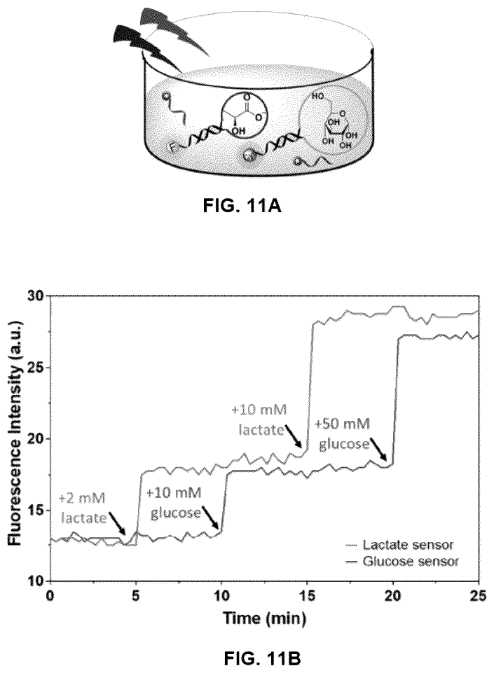

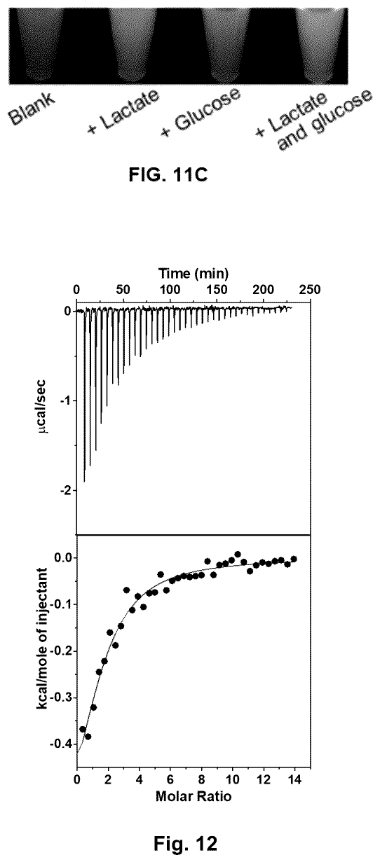

Finally, we tested if we could achieve the simultaneous detection of both lactate and glucose. The glucose aptamer was labeled with a Cy5 fluorophore and the same quencher-labeled DNA was used. Although our glucose sensor had a slightly different 8 design compared to one reported in the literature it responded to glucose as expected with an instantaneous response (FIGS. 10A and 10B). We mixed the two sensors in the same serum solution and added lactate and glucose, as illustrated in FIG. 11A. When lactate was added, only the FAM channel fluorescence increased, and when glucose was added, only the Cy5 channel fluorescence increased, as shown in FIG. 11B. Both sensors had instantaneous responses. FIG. 11C shows a photograph of the sensor response to lactate and glucose. It was therefore demonstrated that these two aptamers are able to simultaneously detect lactate and glucose in the same sample without cross activities.

We have demonstrated the feasibility of biosensors for metabolites using aptamers instead of enzymes for detection of molecules as simple as lactate. The excellent selectivity of the sensors described herein also permits multiplexed detection of analytes.

In summary, we report here the first DNA aptamers that can bind to L-lactate with a Kd as low as 0.39 mM, which encompasses the physiological concentration range of lactate. Since it does not require other substrates and can directly sense the concentration change by its fast binding, this aptamer will find applications in biomedical diagnosis, and in the food and beverage industry and environmental monitoring. In addition, this work demonstrates the range of analytes to which aptamers can bind. Even for a very simple and low-epitope molecule like L-lactate, high quality aptamers were obtained.

Finally, we have demonstrated multiplex detection of glucose and lactate using aptamer sensors, which would be very useful, for example, for in vivo continuous monitoring of these analytes.

### Materials and Methods

### Reagents

The DNA samples used for the selection and sensing experiments were purchased from Integrated DNA Technologies (Coralville, IA, USA). The sequences are listed in Tables 1 and 2.

TABLE 1
DNA sequences used for the
aptamer selection experiment.

| | Sequences and | SEQ |
|---|---|---|
| | modifications | ID |
| DNA Names | (From 5′ to 3′) | NO. |
| N30 Library | GGAGGCTCTCGGGACGACNNNNNN | 11 |
| | NNNNNNNNNNNNNNNNNNNNNNNN | |
| | GTCGTCCCGATGCTGCAATCGTAA | |
| Forward primer | GGAGGCTCTCGGGACGAC | 12 |
| Reverse primer | TTACGATTGCAGCATCGGGACG | 13 |
| Biotin-reverse | /5Biosg/TTACGATTGCAGCA | 14 |
| primer | TCGGGACG | |
| Biotin- | GTCGTCCCGAGAGCCATA/ | 15 |
| column | 3BioTEG/ | |

Notes: 1) “N” refers to any nucleotide; 2)/5Biosg/indicates biotinylation at the 5′-end; and 3)/3BioTEG/indicates biotinylation at the 3′-end with an extended spacer.

TABLE 2
Other DNA sequences used in this study.

| | Sequences and modifications | SEQ ID |
|---|---|---|
| DNA Names | (From 5′ to 3′) | NO. |
| P5-501 | AATGATACGGCGACCACCGAGATCTACACTAGATCGCAC | 7 |
| (primer) | ACTCTTTCCCTACACGACGCTCTTCCGATCTTTACGATT | |
| | GCAGCATCGGGACG | |
| P7-702 | CAAGCAGAAGACGGCATACGAGATCTAGTACGGTGACT | 8 |
| (primer) | GGAGTTCAGACGTGTGCTCTTCCGATCTGGAGGCTCTC | |
| | GGGACGAC | |
| P7-703 | CAAGCAGAAGACGGCATACGAGATTTCTGCCTGTGACTG | 9 |
| (primer) | GAGTTCAGACGTGTGCTCTTCCGATCTGGAGGCTCTCG | |
| | GGACGAC | |
| P7-704 | CAAGCAGAAGACGGCATACGAGATGCTCAGGAGTGACT | 10 |
| (primer) | GGAGTTCAGACGTGTGCTCTTCCGATCTGGAGGCTCTC | |
| | GGGACGAC | |
| Lac201 | GACGACGAGTAGCGCGTATGAATGCTTTTCTATGGAGT | 1 |
| Aptamer | CGTC | |
| Lac202 | GACGACTGGAGTTTTAACGGCTCTTGCGAAGTCGTC | 2 |
| Aptamer | | |
| FAM-Lac201 | /56-FAM/CTCTCGACGACGAGTAGCGCGTATGAATG | 3 |
| Sensor | CTTTTCTATGGAGTCGTC | |
| FAM-Lac202 | /56-FAM/CTCTCGACGACTGGAGTTTTAACGGCTCTT | 4 |
| Sensor | GCGAAGTCGTC | |
| Cy5-Glucose | /5Cy5/CTCTCGACGACCGTGTGTGTTGCTCTGTAAC | 5 |
| Sensor | AGTGTCCATTGTCGTC | |
| Quencher- | AGTCGTCGAGAG/3IABkFQ/ | 6 |
| cDNA | | |

Sequences P5-501, P7-702, P7-703, and P7-704 are sequencing primers. “56-FAM” represents a 5′ 6-FAM (fluorescein) fluorescent dye attachment. “5CY5” represents a 5′ Cy5™ dye attachment. “3IABKFQ” represents a 3′ lowa Black™ quencher dye attachment.

Thermo Scientific Pierce streptavidin agarose resin was purchased from Fisher Scientific (Ottawa, ON, Canada). Sodium L-lactate, sodium D-lactate, pyruvate, (+)-sodium 3-hydroxybutyrate, dextrose, sucrose, L-alanine, L-cysteine, Glycine, L-serine, sodium chloride, magnesium chloride, potassium chloride, sodium hydroxide, hydrochloric acid, human serum, and Amicon Ultra-0.5™ centrifugal filter unit (3K and 10K) were purchased from Millipore-Sigma (Oakville, ON, Canada). 4-(2-Hydroxyethyl) piperazine-1-ethanesulfonic acid (HEPES) and its sodium salt form were from Biobasic Inc. (Markham, ON, Canada). Micro Bio-Spin™ chromatography columns and SsoFast™ EvaGreen™ supermix were from Bio-Rad. dNTP mix, and Taq DNA polymerase with ThermoPol™ buffer were from New England Biolabs (Ipswich, MA, USA). All buffers and solutions were prepared with Milli-Q™ water and passed through 0.2 μm filters.

### Aptamer Selection

The library design and selection method were based on Stojanovic's previous publications with a few modifications. The details of the procedures are described in our recent publication [46], the entire contents of which are incorporated herein by reference. For L-lactate selection, the SELEX™ buffer contained 500 mM NaCl, 10 mM KCl, 10 mM MgCl₂, and 50 mM pH 7.4 HEPES. The concentration of DNA library and L-lactate used in each round is listed in Table 3.

TABLE 3
Selection conditions for L-lactate binding aptamers.

| Selection round # | N₃₀ library (pmol) | L-lactate (mM) |
|---|---|---|
| 1 | 500 | 1000 |
| 2 | 400 | 1000 |
| 3 | 300 | 1000 |
| 4 | 200 | 1000 |
| 5-10 | 100 | 1000 |
| 11-12 | 100 | 100 |
| 13-14 | 100 | 10 |
| 15-18 | 100 | 1 |
| 19-20 | 100 | 0.5 |

### ITC

To test aptamer binding to lactate, we performed isothermal titration calorimetry (ITC). ITC experiments were performed in MicroCal™ VP-ITC instrument. Lac201 aptamer (300 μM, 2 mL) and L-Lactate (20 mM, 2 mL) were dissolved in the SELEX™ buffer (500 7 mM NaCl, 10 mM MgCl₂, 10 mM KCl, 50 mM HEPES pH 7.5) and the aptamer was annealed by heating to 90° C. followed by slow cooling to room temperature. The samples were then degassed for 10 min prior to measurement. A volume of 1.5 mL aptamer was loaded into the cell chamber and 300 μL target was loaded in the syringe. Except for an initial injection of 0.2 μL over 0.4 sec, 7.5 μL of target was titrated into the cell each time over 15 sec duration for a total of 37 injections at 20° C. The spacing was set for 360 sec between each injection. The thermodynamic values were obtained by fitting the titration curves to a one-site binding model using the Origin software.

### Fluorescent Biosensor Test

The sensing experiments were performed in a 96-well plate using a Tecan Spark™ microplate reader. The stock of 2 μM sensor complex was formed by annealing 200 pmol of the FAM-labeled lactate aptamer (FAM-Lac201 or FAM-Lac202) with 800 pmol of the short quencher-labeled complementary strand (Quencher-DNA) in 100 μL of SELEX™ buffer. In a typical experiment, 100 μL of the sensor sample with 20 nM aptamer strand was used. The background fluorescence of the sensor was equilibrated and monitored for 5 min before 2 μL of L-lactate or other biomolecules was added. The FAM signaling kinetics were continuously monitored for 15 min (Ex: 485 nm; Em: 520 nm). The experiments were run in triplicate and error bars indicate the standard deviations. 2

### Detection in Serum

For sensing in serum, human serum was diluted 10-fold in the SELEX™ buffer (final 10% serum). The same 2 μM sensor complex was diluted in the 10% serum to a final of 20 nM sensor complex. The background fluorescence of the sensor in serum was stabilized and monitored for 5 min before adding 2 μL lactate. The signaling kinetics were continuously monitored for another 15 min. For multiplex detection, each sample contained both 20 nM lactate sensor and 20 nM glucose sensor (stock is prepared the same way as lactate sensor described above) in 10% serum. FAM (Ex: 485 nm; Em: 520 nm; gain=50) and Cy5 (Ex: 620 nm; Em: 670 nm; gain=80) were monitored simultaneously for 20 min.

In this study, we define the reaction of the sensor FAM-Lac201, for instance, (designated as F) and Quencher-cDNA (designated as Q) to form the FQ DNA complex according to the following reaction 1:

$$F + Q = FQ . \qquad ( 1 )$$

Similarly, we define the sensing reaction of FQ reacting with lactate (designated as L) according to the following reaction 2:

$$FQ + L = FL + Q . \qquad ( 2 )$$

Consequently, the aptamer binding reaction can be written as the following reaction 3:

$$F + L = F L . \qquad ( 3 )$$

Thus, the Kd of reaction 1 was calculated as:

$${K}_{d 1} = \frac{[ F ] [ Q ]}{[ F Q ]}$$

Similarly, the Kd of reaction 2 was calculated as:

$${K}_{d 2} = \frac{[ FQ ] [ L ]}{[ F L ] [ Q ]}$$

Finally, the Kd of reaction 3 was calculated as

$${K}_{d 3} = {K}_{d 1} {K}_{d 2} = \frac{[ F ] [ Q ]}{[ F Q ]} \frac{[ FQ ] [ L ]}{[ FL ] [ Q ]} = \frac{[ F ] [ L ]}{[ F L ]}$$

We obtained Kd1 from the fitting of FIGS. 5A and 5B directly. Kd2 was calculated based on the apparent Kd2 from the sensor calibration curves in FIGS. 4A-4D. When the lactate concentration is at the apparent Kd value, [FQ] and [FL] had the same concentration. Thus, Kd2= [Kd2 apparent]/[Q]. Since we used 20 nM of the FAM-Lac201 and 80 nM Quencher-cDNA for the sensor, [Q] was 70 nM when lactate was equal to the apparent Kd. Thus, Kd2= [Kd2 apparent]/[Q]=13.5 mM/70 nM=1.93×10⁵. Thus, for the Lac201 aptamer, Kd3=Kd1Kd2=(1.93×10⁵)×(2.01 nM)=0.39 mM.

Similarly, for Lac202, Kd2= [Kd2 apparent]/[Q]=57.5 mM/70 nM=8.21×10⁵. Thus, the Kd3 of the Lac202 aptamer was calculated to be Kd3=Kd1Kd2=(8.21×10⁵)×(2.34 nM)=1.92 mM.

Although the above description includes reference to certain specific embodiments, various modifications thereof will be apparent to those skilled in the art. Any examples provided herein are included solely for the purpose of illustration and are not intended to be limiting in any way. Any drawings provided herein are solely for the purpose of illustrating various aspects of the description and are not intended to be drawn to scale or to be limiting in any way. The scope of the claims appended hereto should not be limited by the preferred embodiments set forth in the above description but should be given the broadest interpretation consistent with the present specification as a whole. The disclosures of all references in the present description herein are incorporated herein by reference in their entirety.

### REFERENCES

1) Pohanka, M., D-Lactic Acid as a Metabolite: Toxicology, Diagnosis, and Detection. BioMed Research International 2020, 2020, 3419034.
2) Pundir, C. S.; Narwal, V.; Batra, B., Determination of Lactic Acid with Special Emphasis on Biosensing Methods: A Review. Biosens. Bioelectron. 2016, 86, 777-790.
3) Zhang, Z.; Xu, X., Lactate Clearance Is a Useful Biomarker for the Prediction of All-cause Mortality in Critically Ill Patients: A Systematic Review and Meta-Analysis *. Critical Care Medicine 2014, 42.
4) Kucherenko, I. S.; Topolnikova, Y. V.; Soldatkin, O. O., Advances in the Biosensors for Lactate and Pyruvate Detection for Medical Applications: A Review. Trac-Trends in Analytical Chemistry 2019, 110, 160-172.
5) Przybyt, M., Lactate Biosensors for Food Industry. Biotechnology and Food Science 2014, 78, 71-88.
6) Nikolaus, N.; Strehlitz, B., Amperometric Lactate Biosensors and Their Application in (Sports) Medicine, for Life Quality and Wellbeing. Microchimica Acta 2008, 160, 15-55.
7) Meng, F.; Peng, M.; Wang, X.; Zhang, G., Lactic Acid Wastewater Treatment by Photosynthetic Bacteria and Simultaneous Production of Protein and Pigments. Environ. Technol. 2022, 43, 163-170.
8) Rathee, K.; Dhull, V.; Dhull, R.; Singh, S., Biosensors Based on Electrochemical Lactate Detection: A Comprehensive Review. Biochemistry and Biophysics Reports 2016, 5, 35-54.
9) Alam, F.; RoyChoudhury, S.; Jalal, A. H.; Umasankar, Y.; Forouzanfar, S.; Akter, N.; Bhansali, S.; Pala, N., Lactate Biosensing: The Emerging Point-of-Care and Personal Health Monitoring. Biosensors & Bioelectronics 2018, 117, 818-829.
10) Bollella, P.; Sharma, S.; Cass, A. E. G.; Antiochia, R., Microneedle-Based Biosensor for Minimally-Invasive Lactate Detection. Biosensors & Bioelectronics 2019, 123, 152-159.
11) Cox, K.; Cocchi, M. N.; Salciccioli, J. D.; Carney, E.; Howell, M.; Donnino, M. W., Prevalence and Significance of Lactic Acidosis in Diabetic Ketoacidosis. Journal of Critical Care 2012, 27, 132-137.
12) Wu, Y.; Dong, Y.; Atefi, M.; Liu, Y.; Elshimali, Y.; Vadgama, J. V., Lactate, a Neglected Factor for Diabetes and Cancer Interaction. Mediators of Inflammation 2016, 2016, 6456018.
13) Duong, H. D.; Rhee, J. I., Ratiometric Fluorescent Biosensors for Glucose and Lactate Using an Oxygen-Sensing Membrane. Biosensors-Basel2021, 11.
14) Forderhase, A. G.; Styers, H. C.; Lee, C. A.; Sombers, L. A., Simultaneous Voltammetric Detection of Glucose and Lactate Fluctuations in Rat Striatum Evoked by Electrical Stimulation of the Midbrain. Analytical and Bioanalytical Chemistry 2020, 412, 6611-6624.
15) Shi, W. H.; Luo, X. J.; Cui, Y., A Tube-Integrated Painted Biosensor for Glucose and Lactate. Sensors 2018, 18.
16) Wang, J.; Chatrathi, M. P.; Collins, G. E., Simultaneous Microchip Enzymatic Measurements of Blood Lactate and Glucose. Anal. Chim. Acta 2007, 585, 11-16.
17) Sato, N.; Okuma, H., Amperometric Simultaneous Sensing System for D-Glucose and L-Lactate Based on Enzyme-Modified Bilayer Electrodes. Anal. Chim. Acta 2006, 565, 250-254.
18) Kaisheva, A.; Atanasov, P.; Gamburzev, S.; Dimcheva, N.; Iliev, I., Amperometric Biosensor for Glucose and Lactate. Sensors and Actuators B-Chemical 1992, 8, 53-57.
19) Wu, L.; Wang, Y.; Xu, X.; Liu, Y.; Lin, B.; Zhang, M.; Zhang, J.; Wan, S.; Yang, C.; Tan, W., Aptamer-Based Detection of Circulating Targets for Precision Medicine. Chem. Rev. 2021, 121, 12035-12105.
20) Roth, A.; Breaker, R. R., The Structural and Functional Diversity of Metabolite-Binding Riboswitches. Annu. Rev. Biochem 2009, 78, 305-334.
21) Alkhamis, O.; Canoura, J.; Bukhryakov, K. V.; Tarifa, A.; DeCaprio, A. P.; Xiao, Y., DNA Aptamer-Cyanine Complexes as Generic Colorimetric Small-Molecule Sensors. Angew. Chem. Int. Ed. 2022, 61, e202112305.
22) Yang, K.-A.; Pei, R.; Stojanovic, M. N., In Vitro Selection and Amplification Protocols for Isolation of Aptameric Sensors for Small Molecules. Methods 2016, 106, 58-65.
23) Li, L.; Xu, S. J.; Yan, H.; Li, X. W.; Yazd, H. S.; Li, X.; Huang, T.; Cui, C.; Jiang, J. H.; Tan, W. H., Nucleic Acid Aptamers for Molecular Diagnostics and Therapeutics: Advances and Perspectives. Angewandte Chemie-International Edition 2021, 60, 2221-2231.
24) Sun, M.; Liu, S. W.; Wei, X. Y.; Wan, S.; Huang, M. J.; Song, T.; Lu, Y.; Weng, X. N.; Lin, Z.; Chen, H. L.; Song, Y. L.; Yang, C. Y., Aptamer Blocking Strategy Inhibits Sars-Cov-2 Virus Infection. Angewandte Chemie-International Edition 2021, 60, 10266-10272.
25) Dauphin-Ducharme, P.; Yang, K.; Arroyo-Currás, N.; Ploense, K. L.; Zhang, Y.; Gerson, J.; Kurnik, M.; Kippin, T. E.; Stojanovic, M. N.; Plaxco, K. W., Electrochemical Aptamer-Based Sensors for Improved Therapeutic Drug Monitoring and High-Precision, Feedback-Controlled Drug Delivery. ACS Sensors 2019, 4, 2832-2837.
26) Yang, K.-A.; Barbu, M.; Halim, M.; Pallavi, P.; Kim, B.; Kolpashchikov, D. M.; Pecic, S.; Taylor, S.; Worgall, T. S.; Stojanovic, M. N., Recognition and Sensing of Low-Epitope Targets Via Ternary Complexes with Oligonucleotides and Synthetic Receptors. Nature Chemistry 2014, 6, 1003-1008.
27) Nakatsuka, N.; Yang, K.-A.; Abendroth, J. M.; Cheung, K. M.; Xu, X.; Yang, H.; Zhao, C.; Zhu, B.; Rim, Y. S.; Yang, Y.; Weiss, P. S.; Stojanović, M. N.; Andrews, A. M., Aptamer-Field-Effect Transistors Overcome Debye Length Limitations for Small-Molecule Sensing. Science 2018, 362, 319-324.
28) Nutiu, R.; Li, Y., In Vitro Selection of Structure-Switching Signaling Aptamers. Angew. Chem. Int. Ed. 2005, 44, 1061-1065.
29) Yang, K.-A.; Chun, H.; Zhang, Y.; Pecic, S.; Nakatsuka, N.; Andrews, A. M.; Worgall, T. S.; Stojanovic, M. N., High-Affinity Nucleic-Acid-Based Receptors for Steroids. ACS Chemical Biology 2017, 12, 3103-3112.
30) Yu, H.; Luo, Y.; Alkhamis, O.; Canoura, J.; Yu, B.; Xiao, Y., Isolation of Natural DNA Aptamers for Challenging Small-Molecule Targets, Cannabinoids. Anal. Chem. 2021, 93, 3172-3180.
31) Huang, P.-J. J.; Liu, J., Selection of Aptamers for Sensing Caffeine and Discrimination of Its Three Single Demethylated Analog. Anal. Chem. 2022, in press, 3142-3149.
32) Lyu, C.; Khan, I. M.; Wang, Z., Capture-Selex for Aptamer Selection: A Short Review. Talanta 2021, 229, 122274.
33) Luo, Y.; Jin, Z.; Wang, J.; Ding, P.; Pei, R., The Isolation of a DNA Aptamer to Develop a Fluorescent Aptasensor for the Thiamethoxam Pesticide. Analyst 2021, 146, 1986-1995.
34) Jenison, R. D.; Gill, S. C.; Pardi, A.; Polisky, B., High-Resolution Molecular Discrimination by RNA. Science (Washington, DC, United States) 1994, 263, 1425-1429.
35) Zuker, M., Mfold Web Server for Nucleic Acid Folding and Hybridization Prediction. Nucleic Acids Res. 2003, 31, 3406-3415.
36) Nutiu, R.; Li, Y., Structure-Switching Signaling Aptamers. J. Am. Chem. Soc. 2003, 125, 4771-4778.
37) Bonaventura, J. M.; Sharpe, K.; Knight, E.; Fuller, K. L.; Tanner, R. K.; Gore, C. J., Reliability and Accuracy of Six Hand-Held Blood Lactate Analysers. J Sports Sci Med 2015, 14, 203-214.
38) Hu, J.; Easley, C. J., A Simple and Rapid Approach for Measurement of Dissociation Constants of DNA Aptamers against Proteins and Small Molecules Via Automated Microchip Electrophoresis. Analyst 2011, 136, 3461-3468.
39) Peyrin, E., Enantioselective Properties of Nucleic Acid Aptamer Molecular Recognition Elements. In Chiral Recognition in Separation Methods: Mechanisms and Applications, Berthod, A., Ed. Springer Berlin Heidelberg: Berlin, Heidelberg, 2010; pp 275-288.
40) Feagin, T. A.; Olsen, D. P. V.; Headman, Z. C.; Heemstra, J. M., High-Throughput Enantiopurity Analysis Using Enantiomeric DNA-Based Sensors. J. Am. Chem. Soc. 2015, 137, 4198-4206.
41) Ewaschuk, J. B.; Naylor, J. M.; Zello, G. A., D-Lactate in Human and Ruminant Metabolism. The Journal of Nutrition 2005, 135, 1619-1625.
42) Laffel, L., Ketone Bodies: A Review of Physiology, Pathophysiology and Application of Monitoring to Diabetes. Diabetes/Metabolism Research and Reviews 1999, 15, 412-426.
43) Psychogios, N.; Hau, D. D.; Peng, J.; Guo, A. C.; Mandal, R.; Bouatra, S.; Sinelnikov, I.; Krishnamurthy, R.; Eisner, R.; Gautam, B.; Young, N.; Xia, J.; Knox, C.; Dong, E.; Huang, P.; Hollander, Z.; Pedersen, T. L.; Smith, S. R.; Bamforth, F.; Greiner, R.; McManus, B.; Newman, J. W.; Goodfriend, T.; Wishart, D. S., The Human Serum Metabolome. PLOS ONE 2011, 6, e16957.
44) Kim, J.; Campbell, A. S.; de Ávila, B. E.-F.; Wang, J., Wearable Biosensors for Healthcare Monitoring. Nat. Biotechnol. 2019, 37, 389-406.
45) Huang, P.-J. J.; Liu, J., Selection of Aptamers for Sensing Caffeine and Discrimination of Its Three Single Demethylated Analog. Anal. Chem. 2022, 3142-3149.
46) Hu, J.; Easley, C. J., A Simple and Rapid Approach for Measurement of Dissociation Constants of DNA Aptamers against Proteins and Small Molecules Via Automated Microchip Electrophoresis. Analyst 2011, 136, 3461-3468.

1. A DNA aptamer for binding to L-lactate, the aptamer comprising a nucleic acid sequence according to SEQ ID NO: 1 or SEQ ID NO: 2, or a sequence having at least 80% homology thereto.

2. The DNA aptamer of claim 1, wherein the aptamer has a nucleic acid sequence according to SEQ ID NO: 1 or SEQ ID NO: 2.

3. A use of the aptamer of claim 1 in a method for detecting or monitoring the presence of L-lactate in a medium.

4. The use of claim 3, wherein the medium is blood.

5. A biosensor for detecting the presence of L-lactate, the biosensor comprising the aptamer of claim 1.

6. The biosensor of claim 5, wherein the aptamer includes a signaling moiety adapted to emit a signal when the aptamer binds to L-lactate.

7. The biosensor of claim 6, wherein the signalling moiety is a fluorescent attachment.

8. The biosensor of claim 6, wherein the signalling moiety is fluorescein.

9. The biosensor of claim 5, wherein the biosensor comprises the nucleic acid sequence according to SEQ ID NO: 3 or SEQ ID NO: 4.

10. A method of detecting or monitoring the presence of L-lactate in a sample, the method comprising adding the biosensor of claim 5 to the sample and monitoring the signal.

11. The method of claim 10, wherein the sample is a biological fluid.

12. The method of claim 10, wherein the sample is blood.

13. A biosensor for detecting the presence of glucose, the biosensor comprising an aptamer, the biosensor comprising the nucleic acid sequence according to SEQ ID NO: 5.

14. (canceled)

15. A method of simultaneously detecting or monitoring the presence of L-lactate and D-glucose in a sample, the method comprising:
providing at least one L-lactate detector, wherein the L-lactate detector comprises the aptamer of claim 1;
providing at least one D-glucose detector, wherein the D-glucose detector comprises an aptamer or a biosensor specific to D-glucose;
adding the at least one L-lactate detector and the at least one D-glucose detector to the sample.

16. The method of claim 15, wherein the L-lactate detector is adapted to emit a first signal when bound to L-lactate and wherein the D-glucose detector is adapted to emit a second signal when bound to D-glucose, wherein the first signal is distinguishable from the second signal.

17. The method of claim 15, wherein the D-glucose detector is a D-glucose biosensor comprising the nucleic acid sequence according to SEQ ID NO: 5.

18. A kit for detecting or monitoring the presence of L-lactate, the kit comprising:
the aptamer of claim 1; and a container for collecting a sample.

19. A kit for simultaneously detecting or monitoring the presence of L-lactate and D-glucose, the kit comprising:
the aptamer of claim 1;
a biosensor or aptamer specific to D-glucose; and,
a container for collecting a sample.

20. A method of simultaneously detecting or monitoring the presence of L-lactate and D-glucose in a sample, the method comprising:
providing at least one L-lactate detector, wherein the L-lactate detector comprises the biosensor of claim 5;
providing at least one D-glucose detector, wherein the D-glucose detector comprises an aptamer or a biosensor specific to D-glucose;
adding the at least one L-lactate detector and the at least one D-glucose detector to the sample.

21. A kit for detecting or monitoring the presence of L-lactate, the kit comprising: the biosensor of claim 5; and a container for collecting a sample.
